(19) United States
(12) Patent Application Publication
(10) Pub. No.: US 2021/0290904 A1
(43) Pub. Date: Sep. 23, 2021

(54) CATHETER ASSEMBLY

(71) Applicant: TERUMO KABUSHIKI KAISHA, Tokyo (JP)

(72) Inventors: Kenji Yokoyama, Kanagawa (JP); Masakazu Miyata, Kanawaga (JP); Kozo Hisamatsu, Kanagawa (JP)

(21) Appl. No.: 17/342,763

(22) Filed: Jun. 9, 2021

Related U.S. Application Data
(63) Continuation of application No. PCT/JP2019/050132, filed on Dec. 20, 2019.

(30) Foreign Application Priority Data
Dec. 26, 2018 (JP) 2018-243557
Dec. 26, 2018 (JP) 2018-243564

(51) Int. Cl. A61M 25/01; A61M 25/00

(52) U.S. Cl. CPC A61M 25/0102; A61M 25/0097

(57) ABSTRACT

A catheter assembly includes a catheter including an expandable and contractible tube provided with a lumen and a contact portion provided in a distal end portion of the tube. A stylet can be inserted in the lumen to make the tube expand in the longitudinal direction by coming into contact with the contact portion. A straight connector is provided on a proximal end side of the catheter which can be coupled to the stylet. The connector includes a male screw portion that is engageable with the stylet. The stylet includes a stylet hub disposed on the proximal end side of the catheter and a coupling member that couples the style hub and the straight connector to each other and is engageable with the male screw portion by rotating independently of the stylet hub.

### CROSS REFERENCE TO RELATED APPLICATIONS

This application is a continuation of PCT Application No. PCT/JP2019/050132, filed Dec. 20, 2019, based on and claiming priority to Japanese Application No. 2018-243557, filed Dec. 26, 2018, and to Japanese Application No. 2018-243564, filed Dec. 26, 2018, all three of which are incorporated herein by reference in their entirety.

### BACKGROUND OF THE INVENTION

The present invention relates to a catheter assembly including a blood transmission hole for transmitting blood to a living body.

Conventionally, treatment using percutaneous cardiopulmonary support (PCPS) has been performed for cardiopulmonary resuscitation, circulatory support, and respiratory support in emergency treatment. The percutaneous cardiopulmonary support is a technique for temporarily assisting or substituting for a cardiopulmonary function using an extracorporeal circulatory device. The extracorporeal circulatory device is also used for open heart surgery.

The extracorporeal circulatory device includes an extracorporeal circulation circuit including a centrifugal pump, an artificial lung, a blood removal channel, a blood transmission channel, and the like, performs gas exchange for the blood that has been removed, and then transmits the resultant blood into the blood transmission channel. In this context, U.S. Pat. No. 7,748,275B2 describes an example of a circulation circuit of an extracorporeal circulatory device.

In such a circulation circuit, a catheter is used that includes a blood transmission hole (outflow hole) for transmitting the blood after gas exchange to a desired position of a living body. The catheter is placed at a predetermined position in the living body to be used for extracorporeal circulation. In addition, a catheter that can expand and contract in a radial direction and axial direction (longitudinal direction) to be easily arranged at the predetermined position is used. Furthermore, the catheter may be used together with a medical device such as a stylet for restricting the shape of the catheter moving in a lumen of a living body. The stylet has a function of contracting the catheter in the radial direction, by applying tensile force in the axial direction of the catheter to expand the catheter when the catheter is inserted in the lumen of the living body. The catheter and stylet may be fixed through engagement between spiral shaped members such as a screw.

Under this condition, an attempt to engage the spiral shaped members of the catheter and the stylet through rotation of the stylet in a state where the tensile force is applied by the stylet in the longitudinal direction of the catheter, involves a risk that a distal end of the catheter might rotate to follow the stylet to be twisted. When such twisting occurs, a load due to the rotation of the catheter continues to be applied, resulting in potential deterioration of insertability of the catheter for percutaneous insertion.

### SUMMARY OF THE INVENTION

In view of this, an object of the present invention is to provide a catheter assembly with which twisting occurring in a catheter when the catheter and a stylet are connected to each other is prevented or suppressed, so that deterioration of the insertability of the catheter for percutaneous insertion is prevented or suppressed.

A catheter assembly achieving the above object may include: a catheter including a tube that is formed to have an elongated shape, is provided with a lumen in which blood is flowable, and is expandable and contractible, and to have a first contact portion configured to be contacted in a manner that enables the tube to expand in a longitudinal direction, the first contact portion being provided in a distal end portion of the tube; a stylet that is configured to be capable of being inserted in the lumen, and enables the tube to expand in the longitudinal direction by coming into contact with the first contact portion; and a connector capable of coupling with the stylet, the connector being mounted on a proximal end side of the tube, wherein the connector includes a first engagement portion engageable with the stylet, and wherein the stylet includes a hub disposed on the proximal end side of the catheter and a coupling member that couples the hub and the connector to each other, the coupling member being engageable with the first engagement portion by rotating independently of the hub.

In the catheter assembly with the configuration described above, the stylet is configured to receive the coupling member which is engageable with the first engagement portion by rotating independently of the hub. With this configuration, even when the coupling member of the stylet rotates, no or almost no rotation is transmitted to the hub of the stylet. Thus, twisting of the catheter can be prevented or suppressed. Thus, deterioration of the insertability of the catheter for percutaneous insertion can be prevented or suppressed.

### BRIEF DESCRIPTION OF DRAWINGS

FIG. 1 is a system diagram illustrating an example of an extracorporeal circulatory device employing a percutaneous catheter provided in a catheter assembly according to an embodiment of the present invention;

FIG. 2 is a side view illustrating a catheter assembly according to a first embodiment separated into a percutaneous catheter and a stylet;

FIG. 3 is a side cross-sectional view illustrating the percutaneous catheter according to the first embodiment;

FIG. 4 is a side view illustrating the catheter assembly according to the first embodiment in a state where the stylet is inserted into the percutaneous catheter;

FIG. 5 is an enlarged cross-sectional view illustrating the percutaneous catheter with the stylet inserted;

FIG. 6 is a diagram illustrating a distal end tip;

FIG. 7 is a side cross-sectional view illustrating a straight connector and the stylet coupled on a proximal end side of the catheter assembly according to the first embodiment;

FIG. 8 is a side cross-sectional view illustrating a straight connector and a stylet coupled on a proximal end side of a catheter assembly according to a first modification;

FIG. 9 is a side cross-sectional view illustrating an example of the straight connector and the stylet configured on the proximal end side of the catheter assembly according to the first modification;

FIG. 10 is a partial enlarged view of a portion of FIG. 9;

FIG. 11 is a side cross-sectional view illustrating another example of the straight connector and the stylet configured on the proximal end side of the catheter assembly according to the first modification;

FIG. 12 is a partial enlarged view of a portion of FIG. 11;

FIG. 13 is a side cross-sectional view illustrating a straight connector and a stylet coupled on a proximal end side of a catheter assembly according to a second modification;

FIG. 14 is a plan view illustrating a catheter assembly according to a second embodiment separated into a percutaneous catheter and a stylet;

FIG. 15 is a side cross-sectional view illustrating the percutaneous catheter according to the second embodiment; and

FIG. 16 is a side view illustrating the catheter assembly according to the second embodiment in a state where the stylet is inserted into the percutaneous catheter.

### DETAILED DESCRIPTION OF THE PREFERRED EMBODIMENTS

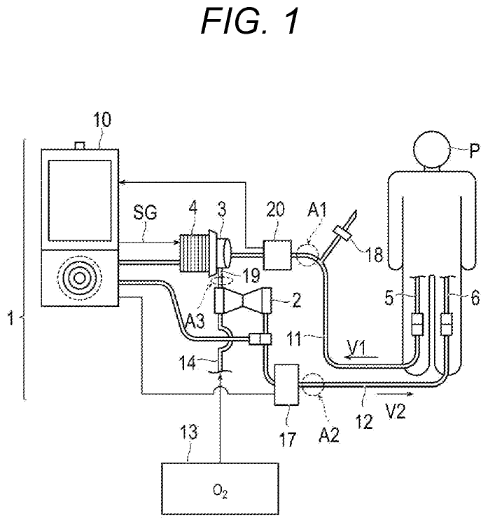

Hereinafter, embodiments of the present invention will be described with reference to the attached drawings. FIG. 1 is a diagram illustrating an example of an extracorporeal circulatory device employing a catheter provided in a catheter assembly according to an embodiment of the present invention. The extracorporeal circulatory device can be used, for example, in percutaneous cardiopulmonary support (PCPS), which temporarily assists or substitutes for heart and lung functions until the cardiac function of a patient with a weakened heart is restored.

An extracorporeal circulatory device 1 can be used for veno-arterial (VA) procedures. In the venous-arterial procedure (VA), a pump is actuated to remove blood from a patient's vein (for example, a large vein), an artificial lung 2 exchanges gas in the blood to oxygenate the blood, and the resultant blood is returned to an artery (for example, aorta). As described above, the extracorporeal circulatory device 1 can be used as a device for assisting the heart and lungs of the patient. Hereinafter, the procedure of removing blood from a patient, performing a predetermined treatment on the blood outside the body, and then transmitting the blood back into the patient's body is referred to as “extracorporeal circulation”.

As illustrated in FIG. 1, the extracorporeal circulatory device 1 includes a circulation circuit for circulating blood. The circulation circuit includes the artificial lung 2, a centrifugal pump 3, a drive motor 4 serving as a driving unit for driving the centrifugal pump 3, a venous catheter (percutaneous catheter for blood removal) 5, and a controller 10 serving as a control unit.

The venous catheter (blood removal) catheter 5 is inserted from the femoral vein, and passes through the inferior vena cava to have the distal end placed in the right atrium. The venous catheter 5 is connected to the centrifugal pump 3 via a blood removal tube (blood removal line) 11. The blood removal tube 11 is a conduit for sending blood.

An arterial catheter (blood transmission catheter) 6 is inserted from the femoral artery.

The drive motor 4 actuates the centrifugal pump 3 based on a command SG from the controller 10. The centrifugal pump 3 sends the blood removed through the blood removal tube 11 into the artificial lung 2, and then returns the resultant blood to a patient P through a blood transmission tube (blood transmission line) 12.

The artificial lung 2 is arranged between the centrifugal pump 3 and the blood transmission tube 12. The artificial lung 2 performs gas exchange for blood (addition of oxygen and/or removal of carbon dioxide). As the artificial lung 2, for example, a membrane type artificial lung can be used, and particularly preferably, a hollow fiber membrane type artificial lung can be used. Oxygen gas is supplied to the artificial lung 2 from an oxygen gas supply unit 13 through a tube 14. The blood transmission tube 12 is a conduit connecting the artificial lung 2 and the arterial catheter 6 to each other.

As the blood removal tube 11 and the blood transmission tube 12, for example, a conduit made of highly transparent, elastically deformable, and flexible synthetic resin, such as a vinyl chloride resin or silicone rubber, can be used. In the blood removal tube 11, the blood, which is liquid, flows in a V1 direction, and in the blood transmission tube 12, the blood flows in a V2 direction.

In the circulation circuit illustrated in FIG. 1, a detection sensor 20 is arranged at an intermediate portion of the blood removal tube 11. A fastening clamp 17 is arranged at an intermediate portion of the blood transmission tube 12.

When bubbles are mixed in the circuit during extracorporeal circulation due to an erroneous operation of a three-way stopcock 18, the tube being damaged, or the like, the detection sensor 20 detects the mixed bubbles by means of ultrasonic waves. When detecting bubbles in the blood sent into the blood removal tube 11, the detection sensor 20 sends a detection signal to the controller 10. Based on this detection signal, the controller 10 issues an alert using an alarm, and reduces the rotation speed of the centrifugal pump 3 or stops the centrifugal pump 3. Further, the controller 10 commands the fastening clamp 17 to immediately close the blood transmission tube 12. This prevents bubbles from being sent into the body of the patient P. In this manner, the controller 10 controls the operation of the extracorporeal circulatory device 1 to prevent bubbles from entering the body of the patient P.

A pressure sensor is provided in the tube 11 (12, 19) of the circulation circuit of the extracorporeal circulatory device 1. The pressure sensor can be, for example, provided at at least one of an attachment position A1 of the blood removal tube 11, an attachment position A2 of the blood transmission tube 12 of the circulation circuit, and an attachment position A3 of a connection tube 19 connecting the centrifugal pump 3 and the artificial lung 2 to each other. The pressure sensor measures the pressure inside of each of the tubes 11, 12, and 19 while the extracorporeal circulatory device 1 is performing extracorporeal circulation for the patient P. The mounting position of the pressure sensor is not limited to the attachment positions A1, A2, and A3, and may be attached at any position in the circulation circuit.

### First Embodiment

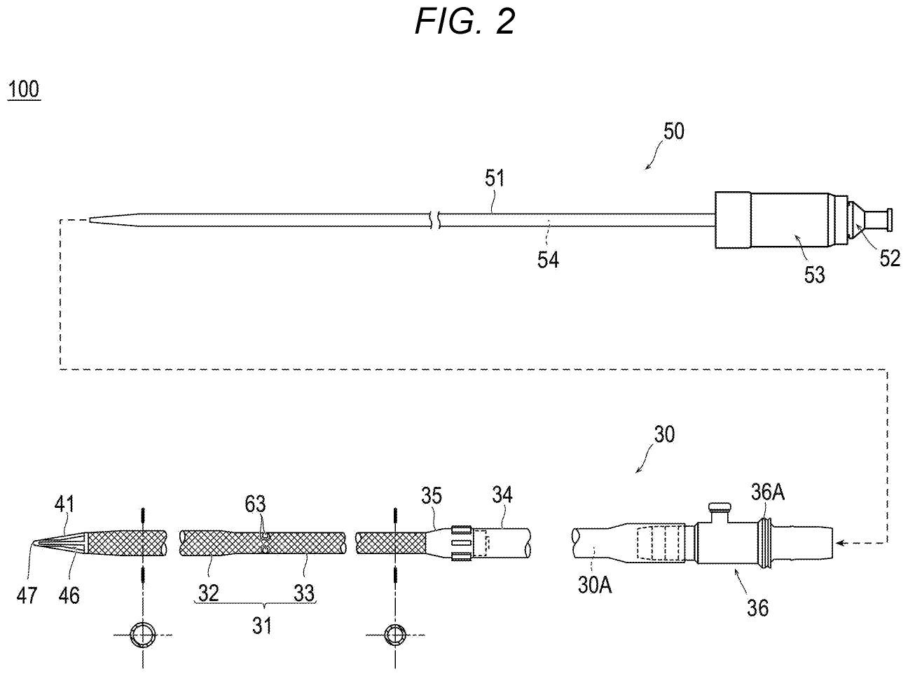

Next, a catheter assembly 100 according to the present embodiment will be described. FIGS. 2 to 7 are diagrams used for a description on the catheter assembly 100 according to a first embodiment. The catheter assembly 100 according to the present embodiment includes a catheter 30 and a stylet 50. The catheter 30 is used as the venous catheter (blood removal catheter) 5 in FIG. 1.

As illustrated in FIG. 2, the catheter 30 according to the present embodiment includes a catheter tube 31 (corresponding to a “tube”) having a side hole 63 and a distal end tip 41 that is arranged at the distal end of the catheter tube 31 and includes through holes 46 and 47. The catheter 30 includes a clamp tube 34 arranged on the proximal end side of the catheter tube 31, a catheter connector 35 connecting the catheter tube 31 and the clamp tube 34 to each other, and a straight connector 36 (corresponding to a “connector”) mounted to a proximal end of clamp tube 34.

In the present specification, the side to be inserted into the living body is referred to as a “distal end” or “distal end side”, and the side of the hand of the operator performing an operation is referred to as a “proximal end” or “proximal end side”. A distal end portion refers to a certain range including the distal end (distal most end) and the periphery thereof. A proximal end portion refers to a certain range including the proximal end (proximal most end) and the periphery thereof.

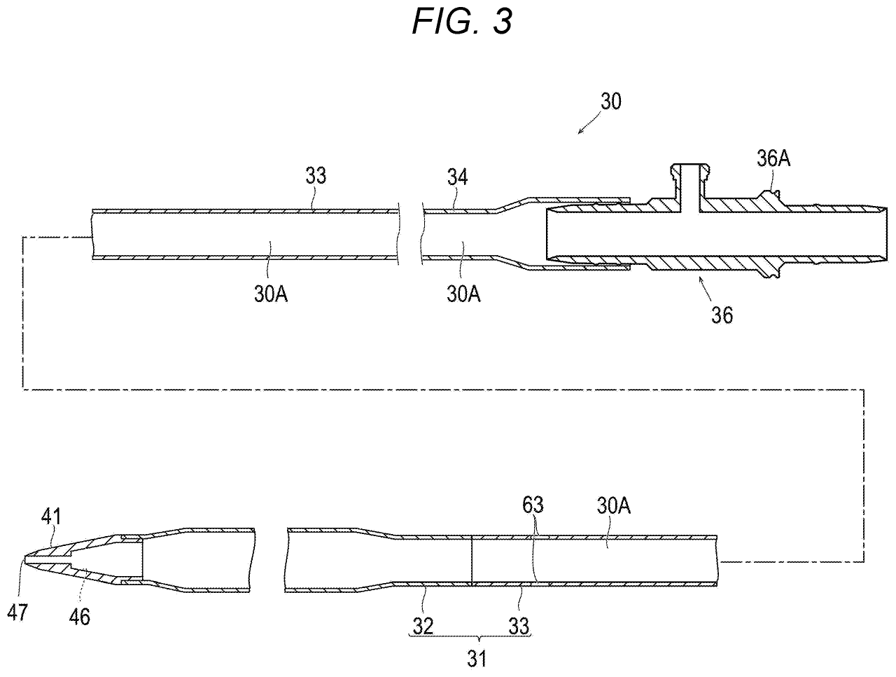

The catheter 30 has a lumen 30A formed therethrough from the distal end to the proximal end as illustrated in FIG. 3. The through holes 46 and 47 of the distal end tip 41 and the side hole 63 of the catheter tube 31 are arranged in different blood removal targets in the living body so that blood can be efficiently removed.

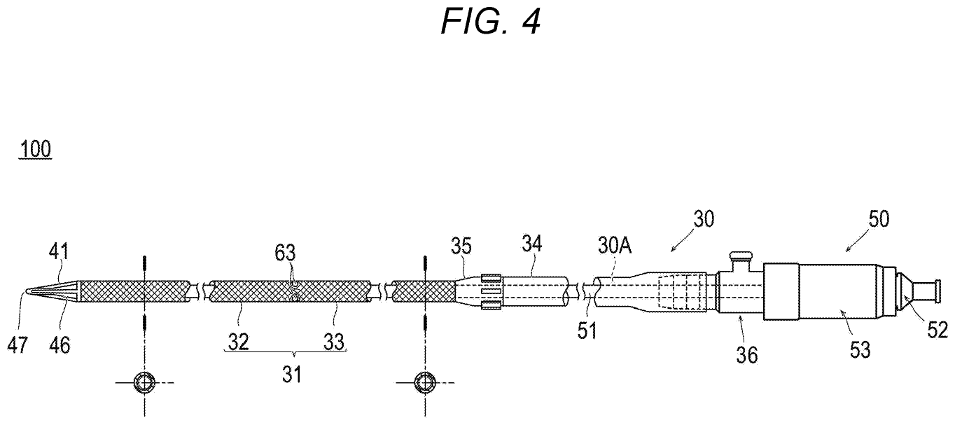

As illustrated in FIG. 4, the stylet 50 is used for inserting the catheter 30 into the living body. The stylet 50 is inserted into the lumen 30A of the catheter 30, and the catheter 30 and the stylet 50 in a pre-integrated state are inserted into the living body. The lumen 30A is configured to enable blood to flow therethrough. How the catheter 30 is used will be described later.

The catheter tube 31 is configured to have an elongated shape, and to be expandable and contractible. The catheter tube 31 includes a first tube 32 and a second tube 33 located on the proximal end side of the first tube 32 as illustrated in FIG. 2. The first tube 32 is configured to have higher elasticity than the second tube 33.

The first tube 32 is configured to have an outer diameter and an inner diameter that are larger than those of the second tube 33. The first tube 32 and the second tube 33 are integrally formed, and are configured to have a substantially uniform thickness in a natural, relaxed state.

The first tube 32 and the second tube 33 are configured to have lengths required for the through holes 46 and 47 of the distal end tip 41 and the side hole 63 to be arranged at the desired blood removal targets. The length of the first tube 32 can be, for example, 20 to 40 cm, and the length of the second tube 33 can be, for example, 20 to 30 cm.

The side hole 63 is a hole that is formed through the side surface of the second tube 33 and is opened to be in communication with the lumen 30A of the catheter 30. The side hole 63 functions as a hole for blood removal. The side hole 63 preferably includes a plurality of holes. With such a configuration, even when any of the holes is closed by being adsorbed on the blood vessel wall, the blood removal can be implemented through the other holes, so that the extracorporeal circulation can be stably implemented.

In the present embodiment, the blood removal targets are two portions that are the right atrium and the inferior vena cava. The catheter 30 is inserted and placed in the living body to make the through holes 46 and 47 of the distal end tip 41 arranged in the right atrium.

In a state where the through holes 46 and 47 and the side hole 63 are arranged in the blood removal targets, the first tube 32 is placed in the inferior vena cava which is a relatively thick blood vessel, and the second tube 33 is placed in the femoral vein which is a relatively thin blood vessel.

When the stylet 50 is inserted into the lumen 30A of the catheter 30, as illustrated in FIG. 4, the highly elastic first tube 32 expands in the axial direction to have the outer diameter and the inner diameter reduced. Then, the outer diameter and inner diameter of the first tube 32 become substantially the same as the outer diameter and inner diameter of the second tube 33. The catheter 30 is inserted into the living body in a state where the first tube 32 is expanded in the axial direction to have the outer diameter reduced, and thus can be inserted in a minimally invasive manner.

When the stylet 50 is removed from the lumen 30A of the catheter 30 after the catheter 30 has been placed in the living body, the first tube 32 is contracted in the axial direction to have the outer diameter and the inner diameter increased as illustrated in FIG. 2. The first tube 32 is placed in the inferior vena cava which a relatively thick blood vessel, and thus can have a large outer diameter.

Here, the pressure loss occurring while the blood or the like flows through the first tube 32 may be reduced by increasing the inner diameter of the first tube 32. When the pressure loss is reduced, the flow rate of the blood flowing through the circulation circuit increases. In view of this, to achieve a sufficient blood circulation amount, the inner diameter of the first tube 32 needs to be sufficiently large.

When the thickness is substantially uniform, large inner diameters of the first tube 32 and the second tube 33 directly relate to large outer diameters thereof, imposing a large load on the patient when the catheter 30 is inserted in to the living body. As a result, minimally invasive procedures would be ruined.

In view of the above, the inner diameter of the first tube 32 can be, for example, 9 to 11 mm, and the inner diameter of the second tube 33 can be, for example, 4 to 8 mm. The thicknesses of the first tube 32 and the second tube 33 can be, for example, 0.3 to 0.5 mm.

As illustrated in FIGS. 2 and 3, the distal end portion and the proximal end portion of the first tube 32 form tapered portions to have diameters gradually decreasing respectively toward the distal end portion and the proximal end portion from the center of the first tube 32 in the longitudinal direction. This configuration facilitates continuous transition between the inner diameters of the distal end and the proximal end of the first tube 32 and the inner diameters of the distal end tip 41 arranged on the distal end side and of the second tube 33 arranged on the proximal end side.

Next, a specific configuration of the catheter tube 31 will be described.

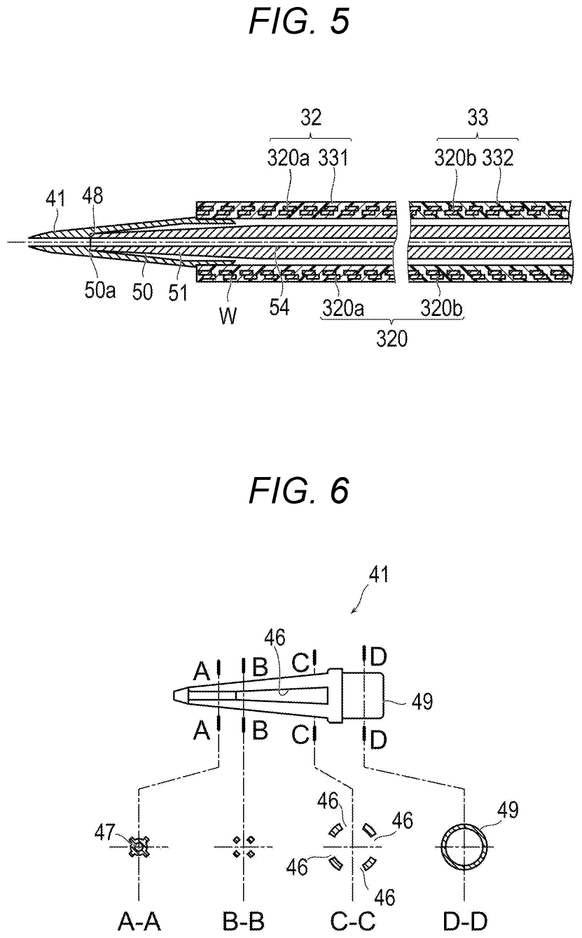

As illustrated in FIG. 5, the catheter tube 31 has a tubular reinforcing body 320 obtained by braiding wires W in a mesh pattern in an intersecting manner, as well as a first resin layer 331 and a second resin layer 332 provided to cover the reinforcing body 320.

The first tube 32 may include a distal end portion 320a of the reinforcing body 320 and the first resin layer 331, and the second tube 33 may include a proximal end portion 320b of the reinforcing body 320 and the second resin layer 332.

By braiding the plurality of the wires W, a large number of gap portions or opening portions are formed in the reinforcing body 320. The relationship among the plurality of gap portions in size is not particularly limited.

The second resin layer 332 is formed to cover the inner circumference surface of the opening portion of the reinforcing body 320. Thus, the wires can be prevented from being exposed from the inner circumference surface of the side hole 63. The maximum length of the opening portion can be about 2 to 3 mm.

The wires W forming the reinforcing body 320 are made of a shape memory material such as a known shape memory metal or shape memory resin. As the shape memory metal, for example, a titanium-based (such as Ti—Ti, Ti—Pd, or Ti—Nb—Sn) alloy or a copper-based alloy can be used. As the shape memory resin, for example, acrylic resin, a transisoprene polymer, polynorbornene, a styrene-butadiene copolymer, or polyurethane can be used.

In the present embodiment, the wires W forming the reinforcing body 320 have a rectangular cross-sectional shape. However, this should not be construed in a limiting sense, and a shape other than the above such as square, circular, and elliptical may be employed. When the cross-sectional shape is circular, the diameter of the wire W can be, for example, 0.1 mm to 0.2 mm.

The first resin layer 331 forming the first tube 32 is made of a material softer than that of the second resin layer 332 forming the second tube 33. With this configuration, the first tube 32 can be softer than the second tube 33, enabling higher elasticity.

As the material forming the first resin layer 331, relatively soft known resin can be used, and for example, a urethane, polyurethane, silicon, or vinyl chloride material having a low hardness can be used. As the material forming the second resin layer 332, for example, a urethane, polyurethane, silicon, vinyl chloride material having high hardness can be used.

When urethane or polyurethane is used, the surface may be provided with hydrophilic coating. With this configuration, the catheter tube 31 can have high surface lubricity and can be easily inserted into the living body, whereby operability is improved, and the blood vessel wall can be prevented from being damaged. Furthermore, attachment of blood and proteins is less likely to occur, and thus formation of a blood clot can also be expected to be prevented.

The distal end tip 41 is fixed to the distal end of the first tube 32. As illustrated in FIG. 6, the distal end tip 41 has a tapered shape with the diameter gradually decreasing toward the distal end side. As illustrated in FIG. 6, the distal end tip 41 includes a base portion 49 inserted into the distal end of the first tube 32, the plurality of through holes 46 provided on the side surface, and the through hole 47 provided at the distal end of the distal end tip 41. The through holes 46 and 47 function as holes for blood removal. The through hole 47 of the distal end tip 41 is configured to communicate with the lumen 30A of the catheter 30. The distal end tip 41 can be formed of, for example, hard plastic or the like.

Further, with the hard distal end tip 41 fixed to the distal end portion of the first tube 32, the first tube 32 can be prevented from being crushed during blood removal.

As illustrated in FIG. 5, a flat receiving surface 48 (corresponding to “first contact portion”) that comes into contact with a flat surface 50a of the stylet 50 used before the insertion of the catheter 30 into the living body is formed on the inner side of the distal end tip 41. As will be described later, with the distal end of the stylet 50 being in contact with the receiving surface 48, the catheter 30 expands in the longitudinal direction.

The clamp tube 34 is provided on the proximal end side of the second tube 33. A lumen into which the stylet 50 can be inserted can be provided on the inner side of the clamp tube 34. The clamp tube 34 can be formed using the same material as the catheter tube 31.

The catheter connector 35 connects the second tube 33 and the clamp tube 34 to each other. A lumen into which the stylet 50 can be inserted can be provided on the inner side of the catheter connector 35.

The straight connector 36 is provided on the proximal end side of the catheter 30. The straight connector 36 is sturdily affixed to the proximal end side of the clamp tube 34 (e.g., clamp tube 34 grips a distal end of straight connector 36 so that no sliding or rotation occurs between them). The straight connector 36 has a circular shape in a cross section intersecting the longitudinal direction in the proximal end portion. A lumen into which the stylet 50 can be inserted can be provided on the inner side of the straight connector 36. The straight connector 36 has an outer circumference provided with a male screw portion 36A (corresponding to “first engagement portion”) that can engage with a female screw portion 53C of the stylet 50. The straight connector 36 is configured to be capable of being coupled to the stylet 50 by means of the male screw portion 36A.

(Stylet)

The stylet 50 includes a stylet tube 51 extending in the axial direction, and a stylet hub 52 (corresponding to a “hub”) to which the proximal end of the stylet tube 51 is fixed as illustrated in FIG. 2. The stylet 50 includes a coupling member 53 provided on the distal end side of the stylet hub 52 and coupling the stylet hub 52 and the straight connector 36 to each other.

The stylet tube 51 is a relatively rigid elongated body that extends in the axial direction. The stylet tube 51 is configured to be capable of being inserted into the lumen 30A of the catheter 30. The total length of the stylet tube 51 along the axial direction is configured to be longer than the total length (in its natural, relaxed state) of the catheter 30 along the axial direction. The stylet tube 51 includes a guide wire lumen 54 through which a guide wire (not illustrated) can be inserted (see FIG. 5). The stylet tube 51 is inserted into the living body together with the catheter 30 while being guided by the guide wire. The stylet tube 51 is removed from the catheter 30 by pulling out the stylet hub 52 toward the proximal end side, after the catheter 30 has been placed in the living body.

The distal end of the stylet tube 51 includes the flat surface 50a that comes into contact with the receiving surface 48 of the distal end tip 41 as illustrated in FIG. 5. The stylet tube 51 has a relatively high rigidity, and has a stiffness to enable pushing force toward the distal end side, applied by an operation with the hand, to be transmitted to the distal end tip 41. Thus, the stylet tube 51 has a function of expanding the catheter tube 31 in the longitudinal direction and dilating a narrow blood vessel, by bringing the flat surface 50a of the stylet tube 51 to contact with the receiving surface 48 of the distal end tip 41 and pushing the distal end tip 41 toward the distal end side.

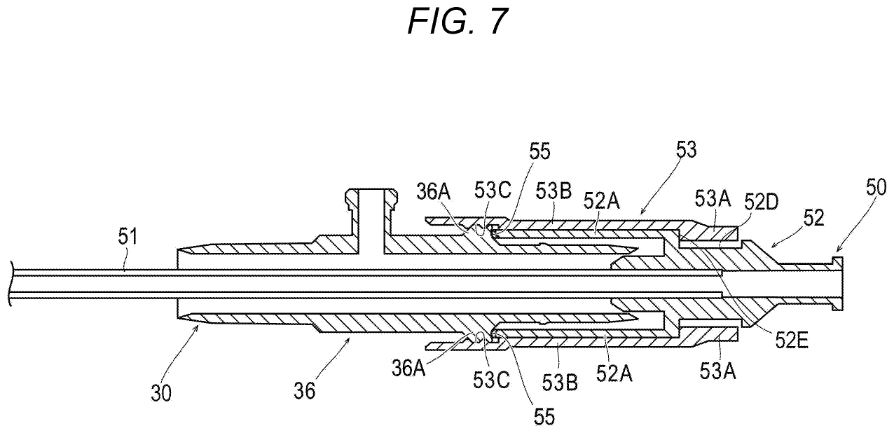

The stylet hub 52 is arranged on the proximal end side of the catheter 30 when the catheter 30 is coupled to the stylet 50. As illustrated in FIG. 7, the stylet hub 52 includes an extending portion 52A extending along a first enclosing portion 53B of the coupling member 53 described later, on the outer side of the stylet hub 52 in the circumferential direction. The extending portion 52A is formed to have a hollow circular shape in a cross section intersecting the longitudinal direction which receives a proximal end of straight connector 36. For clarity, clamp tube 34 which would be connected to the distal end of straight connector 36 is not shown in FIG. 7.

As illustrated in FIG. 7, the coupling member 53 includes the first enclosing portion 53B that extends in the longitudinal direction so as to enclose a space between an attachment portion 53A attached to the stylet hub 52 and the female screw portion 53C (second engagement portion) described later. The attachment portion 53A is rotatably attached to the stylet hub 52. The attachment portion 53A is arranged to be separated from a notched outer surface 52D of the stylet hub 52 in a radial direction. The attachment portion 53A fits into notched surface 52D and applies pressing force on an outer surface 52E of the stylet hub 52, and applies force to fix the stylet hub 52 to the straight connector 36 in the longitudinal direction in response to screwing between the male screw portion 36A and the female screw portion 53C described later. The extending portion 52A extends along the first enclosing portion 53B and comes into contact with the male screw portion 36A (engagement portion). When the male screw portion 36A and the extending portion 52A come into surface contact with each other, a sealing portion 55 sealing the proximal end side of the catheter 30 is formed between the catheter 30 and the stylet 50.

The coupling member 53 includes the female screw portion 53C that is provided on the distal end side of the first enclosing portion 53B and engages with the male screw portion 36A by screwing, and can engage with the male screw portion 36A by rotating independently of the stylet hub 52. The female screw portion 53C is provided on the side opposite to the attachment portion 53A in the longitudinal direction. The female screw portion 53C enables the coupling member 53 to be detachably attached to the straight connector 36 through screwing. The female screw portion 53C corresponds to the second engagement portion in the present specification.

<How Catheter is Used>

Next, how the catheter assembly 100 described above is used will be described. FIG. 2 illustrates a state before the stylet tube 51 of the stylet 50 is inserted into the lumen 30A of the catheter 30, and FIG. 4 illustrates a state after the stylet tube 51 has been inserted into the lumen 30A of the catheter 30.

An operator such as a physician first inserts the stylet tube 51 of the stylet 50 into the lumen 30A of the catheter 30 as illustrated in FIG. 4. As a result, the stylet tube 51 passes through the inside of the straight connector 36, the clamp tube 34, the catheter connector 35, the second tube 33, and the first tube 32 in this order. Then, the flat surface 50a of the stylet tube 51 comes into contact with the receiving surface 48 of the distal end tip 41 (see FIG. 5).

The total length of the stylet tube 51 along the axial direction is configured to be longer than the total length of the catheter 30 along the axial direction as illustrated in FIG. 2. Thus, the distal end tip 41 is pressed toward the distal end side, with the flat surface 50a of the stylet tube 51 being in contact with the receiving surface 48 of the distal end tip 41. As a result, the distal end of the first tube 32 fixed to the distal end tip 41 is pulled toward the distal end side.

Thus, the catheter 30 receives force in the expanding direction, and the first tube 32, which is a relatively elastic portion of the catheter 30, expands in the axial direction. When the first tube 32 expands in the axial direction, the outer diameter of the first tube 32 decrease to be substantially the same as the outer diameter of the second tube 33. With the first tube 32 expanding in the axial direction, the proximal end of the catheter 30 and the stylet hub 52 are fixed to each other. The proximal end of the catheter 30 and the stylet hub 52 are fixed to each other, when the female screw portion 53C and the male screw portion 36A engage with each other due to a manual rotation of the coupling member 53.

Next, the operator inserts the catheter 30 through which the stylet tube 51 is inserted, along a guide wire (not illustrated) that has been inserted into the target portion in the living body in advance. In this processing, by inserting the stylet 50 into the catheter 30, the outer diameter of the first tube 32 becomes substantially the same as the outer diameter of the second tube 33. Therefore, the catheter 30 can be inserted into the living body in a minimally invasive manner. The operator inserts the catheter 30 into the living body until the through holes 46 and 47 of the distal end tip 41 are placed in the right atrium and the side hole 63 is placed in the inferior vena cava, and holds the catheter 30 in this state. In a state where the through holes 46 and 47 and the side hole 63 are arranged in the blood removal targets, the first tube 32 is arranged in the inferior vena cava which is a relative thick blood vessel, and the second tube 33 is arranged in the femoral vein which is a relatively thin blood vessel.

The operator then removes the stylet tube 51 and the guide wire from the catheter 30 by unscrewing coupling member 53 from straight connector 36. In this process, the stylet tube 51 and the guide wire are first pulled out to a position of the straight connector 36 of the catheter 30, temporarily clamped by forceps (not illustrated), and then is completely removed from the catheter 30. When the stylet tube 51 is removed from the lumen 30A of the catheter 30, the catheter 30 is released from force in a direction for causing it to expand in the axial direction, received from the stylet 50. Therefore, the first tube 32 contracts in the axial direction, and the outer diameter and inner diameter of the first tube 32 increase. Thus, the pressure loss in the first tube 32 can be reduced.

Next, the operator connects the straight connector 36 of the catheter 30 to the blood removal tube 11 of the extracorporeal circulatory device 1 illustrated in FIG. 1. Once completion of the connection of the catheter on the blood transmission is confirmed, the forceps of the clamp tube 34 are released and extracorporeal circulation starts.

When the extracorporeal circulation ends, the operator removes the catheter 30 from the blood vessel and performs hemostatic repair on the insertion site through a surgical procedure if necessary.

As described above, the catheter assembly 100 according to the present embodiment includes a catheter 30 and a stylet 50. The catheter 30 includes the catheter tube 31, the receiving surface 48, and the straight connector 36. The catheter tube 31 is formed to have an elongated shape, is provided with the lumen 30A in which the blood can flow, and is configured to be expandable and contractible. The receiving surface 48 is provided in the distal end portion of the catheter tube 31, and enables the catheter tube 31 to expand in the longitudinal direction by the contacting. The straight connector 36 is provided on the proximal end side of the catheter 30 and is configured to be capable of being coupled with the stylet 50. The straight connector 36 includes the male screw portion 36A that can be engaged with the stylet 50. The stylet 50 is configured to be capable of being inserted into the lumen 30A, allows the catheter tube 31 to expand in the longitudinal direction by coming into contact with the receiving surface 48, and includes the stylet hub 52 and the coupling member 53. The stylet hub 52 is arranged on the proximal end side of the catheter 30. The coupling member 53 couples the stylet hub 52 and the straight connector 36 to each other, and is configured to be engageable with the male screw portion 36A by rotating independently of the stylet hub 52.

With this configuration, even when the coupling member 53 rotates, no or almost no rotation of the coupling member 53 is transmitted to the stylet hub 52. Thus, twisting occurring at the distal end of the catheter 30 can be prevented or suppressed when the stylet 50 is connected to the catheter 30. Thus, deterioration of the insertability of the catheter 30 for percutaneous insertion can be prevented or suppressed.

Furthermore, the coupling member 53 includes the attachment portion 53A that is rotatably attached to the stylet hub 52. The attachment portion 53A is arranged to be separated from an outer surface 52D of the stylet hub 52 in a radial direction or a radiation direction. This enables the coupling member 53 to be less likely to be in contact with the stylet hub 52 at the attachment portion 53A. Thus, when the coupling member 53 is rotated relatively with respect to the stylet hub 52, the twisting due to the stylet hub 52 rotating together with the coupling member 53 can be prevented or suppressed.

Further, the coupling member 53 includes the female screw portion 53C that is provided on the opposite side of the attachment portion 53A in the longitudinal direction and is engageable with the male screw portion 36A through screwing. The attachment portion 53A is configured to apply force to fix the stylet hub 52 to the straight connector 36 in the longitudinal direction, in response to screwing between the male screw portion 36A and the female screw portion 53C. With the stylet hub 52 and the coupling member 53 thus configured to be integrated, and provide tightening force in the longitudinal direction and not in the radial direction or the radiation direction, contact therebetween the in radial direction or the radiation direction is not required. Thus, the attachment portion 53A can be arranged to be separated from the stylet hub 52 in the radial direction or the radiation direction as described above, meaning that the contact surface as a result of rotation of one of these can be relatively small. All things considered, twisting due to the rotation of the coupling member 53 can be prevented or suppressed.

### First Modification

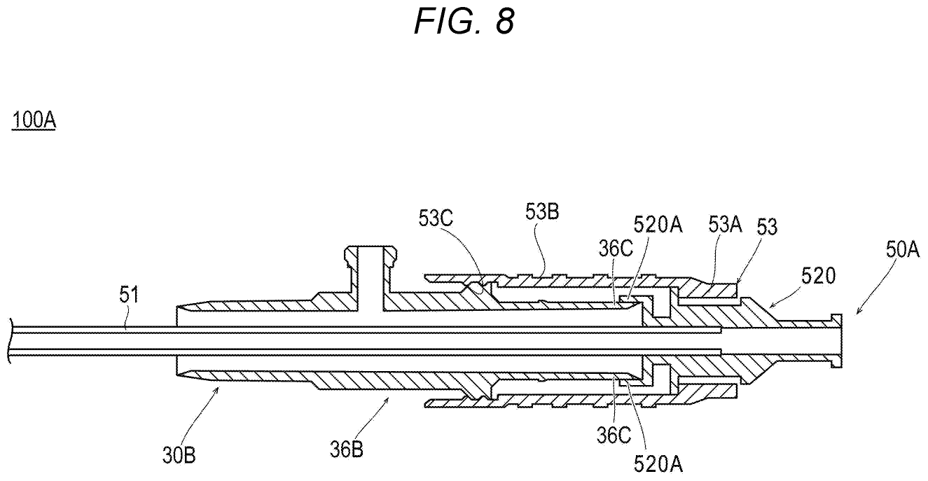

Next, a catheter assembly 100A according to a first modification will be described with reference to FIG. 8. FIGS. 8 to 12 are diagrams used for a description on a main part of the catheter assembly 100A according to the first modification.

As illustrated in FIG. 8, the catheter assembly 100A according to the first modification is different from the first embodiment in that it includes a straight connector 36B forming a portion of a catheter 30B and a stylet hub 520 forming a portion of a stylet 50A. The other configurations are substantially the same as those in the first embodiment, and the description to be redundant herein is basically omitted.

The straight connector 36B has a proximal end portion 36C that is formed more linearly than the proximal end portion of the straight connector 36. The straight connector 36B has a circular shape in a cross section intersecting the longitudinal direction in the proximal end portion 36C, as in the case of the straight connector 36 of the first embodiment.

The stylet hub 520 includes a second enclosing portion 520A capable of surrounding the proximal end portion 36C of the straight connector 36B, on the outer circumference of the straight connector 36B. The second enclosing portion 520A is configured to be capable of coming into contact with the outer side surface of the proximal end portion 36C of the straight connector 36B. The second enclosing portion 520A is configured to have a length, in the longitudinal direction, shorter than that of the extending portion 52A of the stylet hub 52. Further, the second enclosing portion 520A is configured to be separated from the first enclosing portion 53B of the coupling member 53 in the radial direction or the radiation direction. The second enclosing portion 520A corresponds to a second contact portion in the present specification.

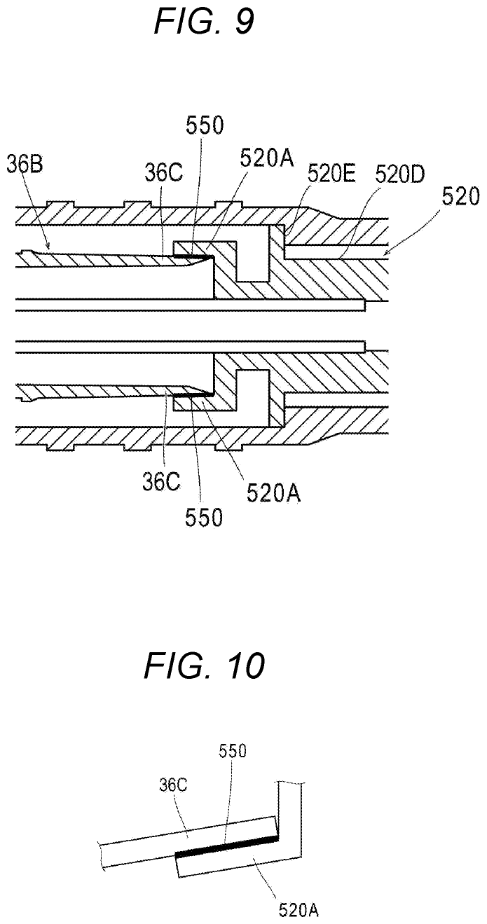

The second enclosing portion 520A and the outer side surface of the proximal end portion 36C of the straight connector 36B may be in surface contact with each other while being inclined in substantially the same manner with respect to the longitudinal direction as illustrated in FIG. 9 and FIG. 10, and thus a sealing portion 550 sealing the proximal end side of the catheter 30 may be formed.

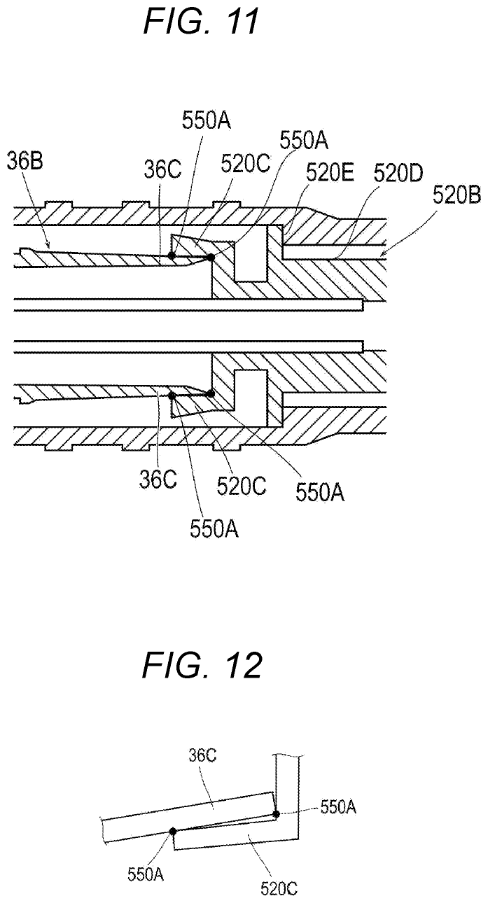

This configuration should not be construed in a limiting sense. For example, a second enclosing portion 520C of the stylet hub 520B and the outer side surface of the proximal end portion 36C of the straight connector 36B may be different from each other in the inclination with respect to the longitudinal direction as illustrated in FIG. 11 and FIG. 12. In this case, at least one of the two is deformed at a portion with different inclinations so that they are in circumferential surface contact with each other around the longitudinal direction. Thus, a sealing portion 550A is formed at the portion of the surface contact. An outer surface 520D of the stylet hubs 520 and 520B illustrated in FIGS. 9 and 11 corresponds to the outer surface 52D, an outer surface 520E corresponds to the outer surface 52E, and the second enclosing portion 520C corresponds to the second contact portion.

As described above, in the first modification, the coupling member 53 is configured to rotate independently of the stylet hub 520, 520B as in the first embodiment. Thus, as in the first embodiment, twisting occurring at the distal end of the catheter 30B can be prevented or suppressed when the stylet 50A is connected to the catheter 30B, and deterioration of the insertability of the catheter 30B for percutaneous insertion can be prevented or suppressed.

Further, the stylet hub 520, 520B includes the second enclosing portion 520A, 520C that comes into contact with the straight connector 36B on the proximal end side of the catheter 30B. The second enclosing portions 520A and 520C are arranged to be separated from the first enclosing portion 53B in the radial direction or the radiation direction. With this configuration, the contact area between the coupling member 53 and the stylet hubs 520 and 520B can be relatively small. Thus, when the coupling member 53 is rotated relatively with respect to the stylet hub 520, 520B, the twisting due to the stylet hub 520, 520B rotating together with the coupling member 53 can be prevented or suppressed.

When the sealing portion 550 is formed with the second enclosing portion 520A being in contact with the outer side surface of the proximal end portion 36C of the straight connector 36B as in the present first modification, no contact occurs on the inner circumference surface of the straight connector 36B. Thus, when surface treatment such as polymer coating is provided on the inner circumference surface, such coating or the like can be prevented from peeling. The same applies to the sealing portion 55 in the first embodiment.

### Second Modification

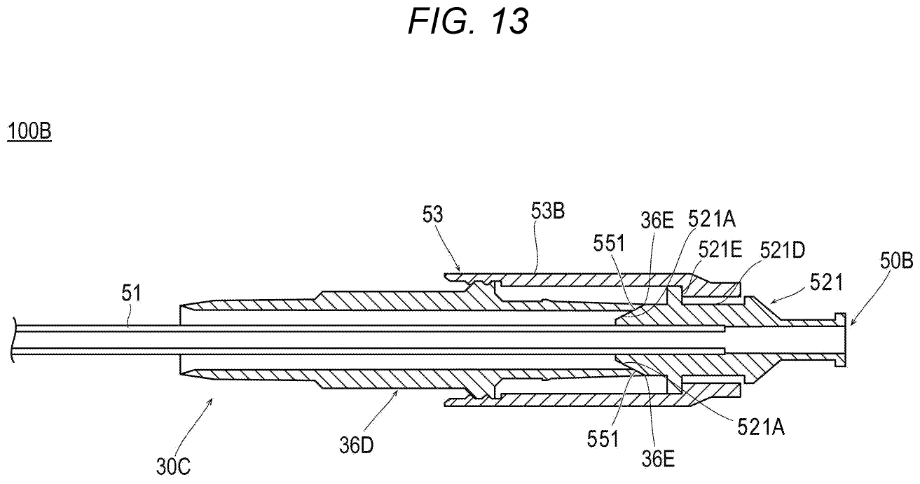

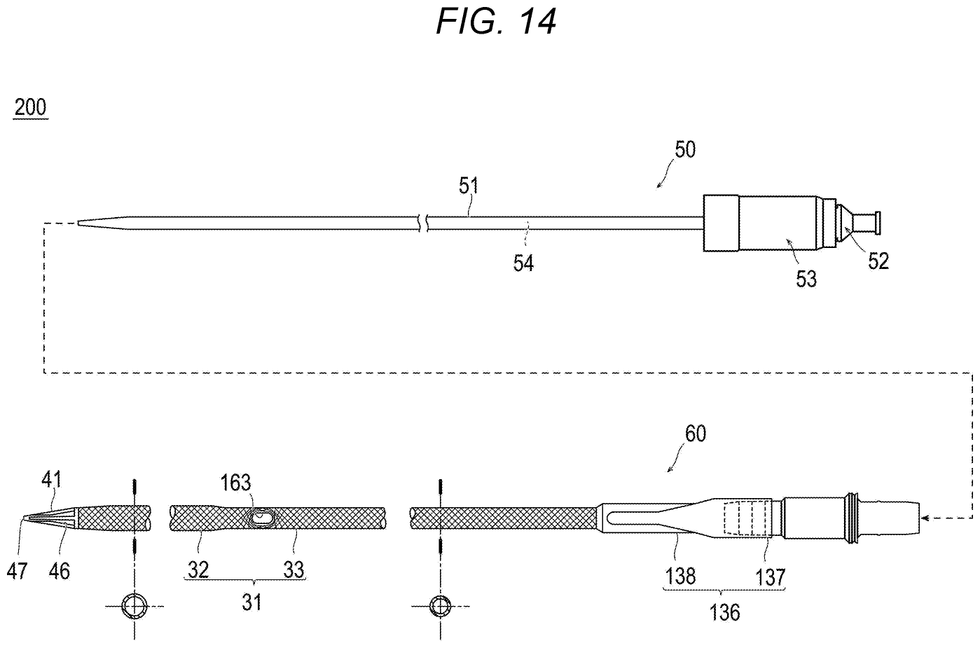

Next, a catheter assembly 100B according to a second modification will be described with reference to FIG. 13. FIG. 13 is a diagram used for a description on a main part of the catheter assembly 100B according to the second modification.

The catheter assembly 100B according to the second modification is, like the first modification, different from the first embodiment in that it includes a straight connector 36D forming a catheter 30C and a stylet hub 521 forming a stylet 50B. Further, the straight connector 36D of the second modification has a proximal end portion provided with a tapered surface 36E formed in a tapered shape. The other configurations are substantially the same as in the first embodiment.

The stylet hub 521 includes a contact portion 521A capable of contacting the tapered surface 36E, and a sealing portion 551 sealing the proximal end side of the catheter 30 is configured by surface contact between the tapered surface 36E and the contact portion 521A. Further, the contact portion 521A is arranged to be separated from the first enclosing portion 53B of the coupling member 53 in the radial direction or the radiation direction as in the first modification. The contact portion 521A corresponds to the second contact portion in the present specification, an outer surface 521D corresponds to the outer surface 52D in the first embodiment, and an outer surface 521E corresponds to the outer surface 52E.

As described above, in the second modification, the coupling member 53 is configured to rotate independently of the stylet hub 521 as in the first embodiment. Thus, as in the first embodiment, twisting occurring at the distal end of the catheter 30C can be prevented or suppressed when the stylet 50B is connected to the catheter 30C, and deterioration of the insertability of the catheter 30C for percutaneous insertion can be prevented or suppressed.

Further, the stylet hub 521 includes the contact portion 521A that comes into contact with the straight connector 36D on the proximal end side of the catheter 30C. The contact portion 521A is arranged to be separated from the first enclosing portion 53B in the radial direction or the radiation direction. Thus, as in the first modification, when the coupling member 53 is rotated relatively with respect to the stylet hub 521, the twisting due to the stylet hub 521 rotating together with the coupling member 53 can be prevented or suppressed. Further, since the coupling member 53 rotates independently of the stylet hub 521, friction between the proximal end side of the catheter 30C and the contact portion 521A of the stylet hub 521 can be prevented. Thus, a coating agent can be prevented from peeling.

### Second Embodiment

Next, a catheter assembly 200 according to a second embodiment of the present invention will be described with reference to FIGS. 14 to 16. FIGS. 14 to 16 are diagrams used for a description on the configuration of the catheter assembly 200 according to the second embodiment. The catheter assembly 200 according to the present embodiment has a percutaneous catheter (hereinafter referred to as “catheter”) 60 different from that in the first embodiment.

This catheter 60 is what is known as a double lumen catheter configured to simultaneously enable both transmission and removal of blood. Thus, in the present embodiment, the catheter 60 is in charge of functions of two catheters, that is, the venous catheter (blood removal catheter) 5 and the arterial catheter (blood transmission catheter) 6 in the extracorporeal circulatory device 1 in FIG. 1.

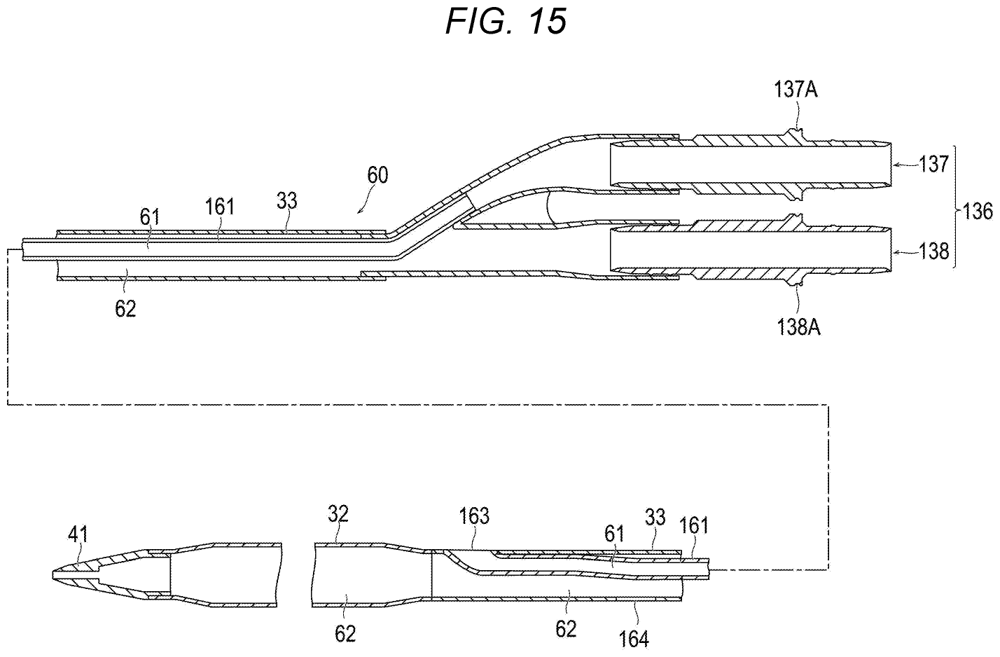

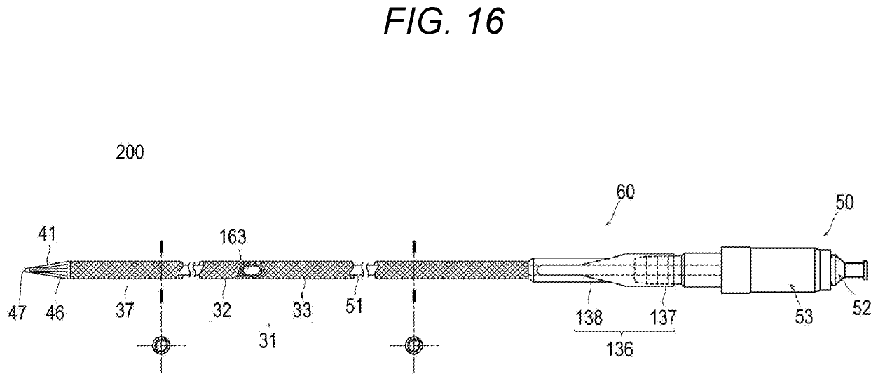

The catheter 60 according to the present embodiment is different from the catheter 30 according to the first embodiment in that a double tube structure is provided as illustrated in FIGS. 15 and 16. In such a structure, a third tube 161 having a first lumen 61 in communication with a blood transmission side hole 163 is provided inside a lumen of the second tube 33.

With the catheter 60, the blood is removed from a vein (vena cava) of the patient with a pump of the extracorporeal circulatory device 1 activated. Thereafter, gas exchange inside the blood is performed with the artificial lung 2 to oxygenate the blood, and then the resultant blood is returned to the vein (vena cava) of the patient again. In this manner, the Veno-Venous (VV) procedure can be performed.

Hereinafter, each configuration of the catheter 60 will be described. The parts common to the first embodiment will be omitted, and characteristic parts of the present embodiment will be described. The parts having the same functions as those in the first embodiment are designated by the same reference numerals, and the description thereof will be omitted.

As illustrated in FIG. 15, the catheter 60 includes the first tube 32, the second tube 33, the distal end tip 41 that is provided at the distal end of the first tube 32 and includes the through holes 46 and 47, and the third tube 161 provided inside the second tube 33.

As illustrated in FIG. 15, the catheter 60 includes the first lumen 61 that functions as the blood transmission channel and a second lumen 62 that functions as the blood removal channel.

The first lumen 61 is formed inside the third tube 161. The second lumen 62 is formed through the first tube 32 and the second tube 33, from the distal end to the proximal end.

The second tube 33 includes the blood transmission side hole 163 in communication with the first lumen 61 that is the blood transmission channel and a blood removal side hole 164 in communication with the second lumen 62 that is the blood removal channel. The blood transmission side hole 163 and the blood removal side hole 164 are formed to have an elliptical shape, but are not limited this shape.

The third tube 161 is configured to be inserted into the second lumen 62 from the proximal end side of the second tube 33 and to be in communication with the blood transmission side hole 163.

The blood transmission side hole 163 is arranged in the blood transmission target in the living body. The blood oxygenated by the artificial lung 2 is transmitted into the living body through the blood transmission side hole 163.

The through holes 46 and 47 of the distal end tip 41 and blood removal side hole 164 are arranged in different blood removal targets in the living body so that blood can be efficiently removed. When the through hole 46, 47 or the blood removal side hole 164 is closed as a result of being adsorbed on a blood vessel wall, blood can be removed through unclosed one of the holes, whereby extracorporeal circulation can be stably performed.

In the present embodiment, the catheter 60 is inserted from the internal jugular vein of the neck, passes through the superior vena cava and the right atrium, to have the distal end placed in the inferior vena cava. The blood transmission target is the right atrium, and the blood removal target includes two portions that are the superior vena cava and the inferior vena cava.

As illustrated in FIGS. 14 and 15, the catheter 60 is inserted, in a state where the stylet 50 is inserted, into the living body and is held once the through holes 46 and 47 of the distal end tip 41 are placed in the inferior vena cava and the blood removal side hole 164 is placed in the internal jugular vein.

As in the first embodiment, the first tube 32 is configured to have an inner diameter that is larger than that of the second tube 33. In a state where the through holes 46 and 47 and the side hole 63 are arranged in the blood removal targets, the first tube 32 is placed in the inferior vena cava which is a relatively thick blood vessel, and the second tube 33 is placed in the femoral vein which is a relatively thin blood vessel.

As illustrated in FIG. 15, a straight connector 136 includes a first straight connector 137 and a second straight connector 138. The first straight connector 137 is configured to communicate with the first lumen 61, and the second straight connector 138 is configured to communicate with the second lumen 62. The straight connector 136 is configured as a Y-shaped Y connector formed with the first straight connector 137 branched from the second straight connector 138. For the sake of illustration, in FIG. 15, the first straight connector 137 and the second straight connector 138 are close to each other, but the first straight connector 137 and the second straight connector 138 are actually arranged to be more separated from each other by an amount larger than that illustrated in FIG. 15.

The first straight connector 137 is coupled to the proximal end portion of the third tube 161. The second straight connector 138 is coaxially coupled to the proximal end portion of the second tube 33. A blood transmission tube (blood transmission line) is connected to the first straight connector 137, and a blood removal tube (blood removal line) is connected to the second straight connector 138. The first straight connector 137 is provided with a male screw portion 137A, and the second straight connector 138 is provided with a male screw portion 138A.

The first tube 32 is configured to function in the same manner as in the first embodiment. When the stylet 50 is inserted into the catheter 60, as illustrated in FIG. 16, the first tube 32 expands to have the outer diameter and the inner diameter reduced. As a result, the catheter 60 can be inserted into the living body in a minimally invasive manner. When the stylet 50 is removed from the catheter 60, the first tube 32 is contracted in the axial direction to have the inner diameter increased as illustrated in FIG. 14. Thus, the pressure loss inside the first tube 32 can be reduced.

As described above, the catheter assembly 200 of the present embodiment has the catheter 60. Thus, both blood removal and blood transmission functions can be provided with a single catheter. As in the first embodiment, twisting occurring at the distal end of the catheter 60 can be prevented or suppressed when the stylet 50 is connected to the catheter 60, and deterioration of the insertability of the catheter 60 for percutaneous insertion can be prevented or suppressed.

The present invention is not limited to the above-described embodiments, and various modifications can be made within the scope of the claims. The embodiments and the like in which the catheter 30 includes the straight connector 36 have been described above. However, the type of the connector is not limited to the straight connector as long as a sealing portion can be formed and the connector can be connected to a mating component.

In a circulation circuit as described in U.S. Pat. No. 7,748,275B2 as a conventional technique, a catheter including a blood transmission hole (outflow hole) for transmitting blood after gas exchange to a desired position of a living body is used. The catheter is moved to the desired lumen of the living body and then connected to another component forming the circulation circuit.

Before the catheter is connected to the other component, the stylet is inserted in the catheter. If the sealing between the catheter and the stylet might is insufficient, the blood might leak. Thus, the sealing therebetween needs to be reliably obtained.

Therefore, in the embodiments described below, a catheter assembly with which the sealing between the catheter and the stylet can be reliably obtained will be described.

A catheter assembly achieving the above object includes: a catheter including a tube that is formed to have an elongated shape, is provided with a lumen in which blood is flowable, and is expandable and contractible, and a contact portion contacting by which enables the tube to expand in a longitudinal direction, the contact portion being provided in a distal end portion of the tube; a stylet that is configured to be capable of being inserted in the lumen, and enables the tube to expand in the longitudinal direction by coming into contact with the contact portion; a connector capable of coupling with the stylet, the connector being provided on a proximal end side of the catheter; and a sealing portion that is provided between the catheter and the stylet and seals the proximal end side of the catheter The connector includes an engagement portion engageable with the stylet, and the stylet includes a hub disposed on the proximal end side of the catheter and a coupling member that couples the hub and the connector to each other and is engageable with the engagement portion.

With the catheter assembly configured as described above, a sealing portion sealing the proximal end side of the catheter is provided between the catheter and the stylet, and thus the sealing therebetween can be reliably obtained.

Hereinafter, embodiments of the present invention will be described with reference to the attached drawings. FIG. 1 is a diagram illustrating an example of an extracorporeal circulatory device employing a catheter provided in a catheter assembly according to an embodiment of the present invention. The extracorporeal circulatory device can be used, for example, in percutaneous cardiopulmonary support (PCPS), which temporarily assists or substitutes for heart and lung functions until the cardiac function of a patient with a weakened heart is restored.

An extracorporeal circulatory device 1 can be used for veno-arterial (VA) procedures. In the venous-arterial procedure (VA), a pump is actuated to remove blood from a patient's vein (for example, a large vein), an artificial lung 2 exchanges gas in the blood to oxygenate the blood, and the resultant blood is returned to an artery (for example, aorta). As described above, the extracorporeal circulatory device 1 can be used as a device for assisting the heart and lungs of the patient. Hereinafter, the procedure of removing blood from a patient, performing a predetermined treatment on the blood outside the body, and then transmitting the blood back into the patient's body is referred to as “extracorporeal circulation”.

As illustrated in FIG. 1, the extracorporeal circulatory device 1 includes a circulation circuit for circulating blood. The circulation circuit includes the artificial lung 2, a centrifugal pump 3, a drive motor 4 serving as a driving unit for driving the centrifugal pump 3, a venous catheter (percutaneous catheter for blood removal) 5, and a controller 10 serving as a control unit.

The venous catheter (blood removal) catheter 5 is inserted from the femoral vein, and passes through the inferior vena cava to have the distal end placed in the right atrium. The venous catheter 5 is connected to the centrifugal pump 3 via a blood removal tube (blood removal line) 11. The blood removal tube 11 is a conduit for sending blood.

An arterial catheter (blood transmission catheter) 6 is inserted from the femoral artery.

The drive motor 4 actuates the centrifugal pump 3 based on a command SG from the controller 10. The centrifugal pump 3 sends the blood removed through the blood removal tube 11 into the artificial lung 2, and then returns the resultant blood to a patient P through a blood transmission tube (blood transmission line) 12.

The artificial lung 2 is arranged between the centrifugal pump 3 and the blood transmission tube 12. The artificial lung 2 performs gas exchange for blood (addition of oxygen and/or removal of carbon dioxide). As the artificial lung 2, for example, a membrane type artificial lung can be used, and particularly preferably, a hollow fiber membrane type artificial lung can be used. Oxygen gas is supplied to the artificial lung 2 from an oxygen gas supply unit 13 through a tube 14. The blood transmission tube 12 is a conduit connecting the artificial lung 2 and the arterial catheter 6 to each other.

As the blood removal tube 11 and the blood transmission tube 12, for example, a conduit made of highly transparent, elastically deformable, and flexible synthetic resin, such as a vinyl chloride resin or silicone rubber, can be used. In the blood removal tube 11, the blood, which is liquid, flows in a V1 direction, and in the blood transmission tube 12, the blood flows in a V2 direction.

In the circulation circuit illustrated in FIG. 1, a detection sensor 20 is arranged at an intermediate portion of the blood removal tube 11. A fastening clamp 17 is arranged at an intermediate portion of the blood transmission tube 12.

When bubbles are mixed in the circuit during extracorporeal circulation due to an erroneous operation of a three-way stopcock 18, the tube being damaged, or the like, the detection sensor 20 detects the mixed bubbles by means of ultrasonic waves. When detecting bubbles in the blood sent into the blood removal tube 11, the detection sensor 20 sends a detection signal to the controller 10. Based on this detection signal, the controller 10 issues an alert using an alarm, and reduces the rotation speed of the centrifugal pump 3 or stops the centrifugal pump 3. Further, the controller 10 commands the fastening clamp 17 to immediately close the blood transmission tube 12. This prevents bubbles from being sent into the body of the patient P. In this manner, the controller 10 controls the operation of the extracorporeal circulatory device 1 to prevent bubbles from entering the body of the patient P.

A pressure sensor is provided in the tube 11 (12, 19) of the circulation circuit of the extracorporeal circulatory device 1. The pressure sensor can be, for example, provided at at least one of an attachment position A1 of the blood removal tube 11, an attachment position A2 of the blood transmission tube 12 of the circulation circuit, and an attachment position A3 of a connection tube 19 connecting the centrifugal pump 3 and the artificial lung 2 to each other. The pressure sensor measures the pressure inside of each of the tubes 11, 12, and 19 while the extracorporeal circulatory device 1 is performing extracorporeal circulation for the patient P. The mounting position of the pressure sensor is not limited to the attachment positions A1, A2, and A3, and may be attached at any position in the circulation circuit.

### Third Embodiment

Next, a catheter assembly 100 according to the present embodiment will be described. FIGS. 2 to 7 are diagrams used for a description on the catheter assembly 100 according to a third embodiment. The catheter assembly 100 according to the present embodiment includes a catheter 30 and a stylet 50. The catheter 30 is used as the venous catheter (blood removal catheter) 5 in FIG. 1.

As illustrated in FIG. 2, the catheter 30 according to the present embodiment includes a catheter tube 31 (corresponding to a “tube”) having a side hole 63 and a distal end tip 41 that is arranged at the distal end of the catheter tube 31 and includes through holes 46 and 47. The catheter 30 includes a clamp tube 34 arranged on the proximal end side of the catheter tube 31, a catheter connector 35 connecting the catheter tube 31 and the clamp tube 34 to each other, and a straight connector 36 (corresponding to a “connector”) mounted to a proximal end of clamp tube 34.

In the present specification, the side to be inserted into the living body is referred to as a “distal end” or “distal end side”, and the side of the hand of the operator performing an operation is referred to as a “proximal end” or “proximal end side”. A distal end portion refers to a certain range including the distal end (distal most end) and the periphery thereof. A proximal end portion refers to a certain range including the proximal end (proximal most end) and the periphery thereof.

The catheter 30 has a lumen 30A formed therethrough from the distal end to the proximal end as illustrated in FIG. 3. The through holes 46 and 47 of the distal end tip 41 and the side hole 63 of the catheter tube 31 are arranged in different blood removal targets in the living body so that blood can be efficiently removed.

As illustrated in FIG. 4, the stylet 50 is used for inserting the catheter 30 into the living body. The stylet 50 is inserted into the lumen 30A of the catheter 30, and the catheter 30 and the stylet 50 in a pre-integrated state are inserted into the living body. The lumen 30A is configured to enable blood to flow therethrough. How the catheter 30 is used will be described later.

The catheter tube 31 is configured to have an elongated shape, and to be expandable and contractible. The catheter tube 31 includes a first tube 32 and a second tube 33 located on the proximal end side of the first tube 32 as illustrated in FIG. 2. The first tube 32 is configured to have higher elasticity than the second tube 33.

The first tube 32 is configured to have an outer diameter and an inner diameter that are larger than those of the second tube 33. The first tube 32 and the second tube 33 are integrally formed, and are configured to have a substantially uniform thickness in a natural, relaxed state.

The first tube 32 and the second tube 33 are configured to have lengths required for the through holes 46 and 47 of the distal end tip 41 and the side hole 63 to be arranged at the desired blood removal targets. The length of the first tube 32 can be, for example, 20 to 40 cm, and the length of the second tube 33 can be, for example, 20 to 30 cm.

The side hole 63 is a hole that is formed through the side surface of the second tube 33 and is opened to be in communication with the lumen 30A of the catheter 30. The side hole 63 functions as a hole for blood removal. The side hole 63 preferably includes a plurality of holes. With such a configuration, even when any of the holes is closed by being adsorbed on the blood vessel wall, the blood removal can be implemented through the other holes, so that the extracorporeal circulation can be stably implemented.

In the present embodiment, the blood removal targets are two portions that are the right atrium and the inferior vena cava. The catheter 30 is inserted and placed in the living body to make the through holes 46 and 47 of the distal end tip 41 arranged in the right atrium.

In a state where the through holes 46 and 47 and the side hole 63 are arranged in the blood removal targets, the first tube 32 is placed in the inferior vena cava which is a relatively thick blood vessel, and the second tube 33 is placed in the femoral vein which is a relatively thin blood vessel.

When the stylet 50 is inserted into the lumen 30A of the catheter 30, as illustrated in FIG. 4, the highly elastic first tube 32 expands in the axial direction to have the outer diameter and the inner diameter reduced. Then, the outer diameter and inner diameter of the first tube 32 become substantially the same as the outer diameter and inner diameter of the second tube 33. The catheter 30 is inserted into the living body in a state where the first tube 32 is expanded in the axial direction to have the outer diameter reduced, and thus can be inserted in a minimally invasive manner.

When the stylet 50 is removed from the lumen 30A of the catheter 30 after the catheter 30 has been placed in the living body, the first tube 32 is contracted in the axial direction to have the outer diameter and the inner diameter increased as illustrated in FIG. 2. The first tube 32 is placed in the inferior vena cava which a relatively thick blood vessel, and thus can have a large outer diameter.

Here, the pressure loss occurring while the blood or the like flows through the first tube 32 may be reduced by increasing the inner diameter of the first tube 32. When the pressure loss is reduced, the flow rate of the blood flowing through the circulation circuit increases. In view of this, to achieve a sufficient blood circulation amount, the inner diameter of the first tube 32 needs to be sufficiently large.

When the thickness is substantially uniform, large inner diameters of the first tube 32 and the second tube 33 directly relate to large outer diameters thereof, imposing a large load on the patient when the catheter 30 is inserted in to the living body. As a result, minimally invasive procedures would be ruined.

In view of the above, the inner diameter of the first tube 32 can be, for example, 9 to 11 mm, and the inner diameter of the second tube 33 can be, for example, 4 to 8 mm. The thicknesses of the first tube 32 and the second tube 33 can be, for example, 0.3 to 0.5 mm.

As illustrated in FIGS. 2 and 3, the distal end portion and the proximal end portion of the first tube 32 form tapered portions to have diameters gradually decreasing respectively toward the distal end portion and the proximal end portion from the center of the first tube 32 in the longitudinal direction. This configuration facilitates continuous transition between the inner diameters of the distal end and the proximal end of the first tube 32 and the inner diameters of the distal end tip 41 arranged on the distal end side and of the second tube 33 arranged on the proximal end side.

Next, a specific configuration of the catheter tube 31 will be described.

As illustrated in FIG. 5, the catheter tube 31 has a tubular reinforcing body 320 obtained by braiding wires W in a mesh pattern in an intersecting manner, as well as a first resin layer 331 and a second resin layer 332 provided to cover the reinforcing body 320.

The first tube 32 may include a distal end portion 320a of the reinforcing body 320 and the first resin layer 331, and the second tube 33 may include a proximal end portion 320b of the reinforcing body 320 and the second resin layer 332.

By braiding the plurality of the wires W, a large number of gap portions or opening portions are formed in the reinforcing body 320. The relationship among the plurality of gap portions in size is not particularly limited.

The second resin layer 332 is formed to cover the inner circumference surface of the opening portion of the reinforcing body 320. Thus, the wires can be prevented from being exposed from the inner circumference surface of the side hole 63. The maximum length of the opening portion can be about 2 to 3 mm.

The wires W forming the reinforcing body 320 are made of a shape memory material such as a known shape memory metal or shape memory resin. As the shape memory metal, for example, a titanium-based (such as Ti—Ti, Ti—Pd, or Ti—Nb—Sn) alloy or a copper-based alloy can be used. As the shape memory resin, for example, acrylic resin, a transisoprene polymer, polynorbornene, a styrene-butadiene copolymer, or polyurethane can be used.

In the present embodiment, the wires W forming the reinforcing body 320 have a rectangular cross-sectional shape. However, this should not be construed in a limiting sense, and a shape other than the above such as square, circular, and elliptical may be employed. When the cross-sectional shape is circular, the diameter of the wire W can be, for example, 0.1 mm to 0.2 mm.

The first resin layer 331 forming the first tube 32 is made of a material softer than that of the second resin layer 332 forming the second tube 33. With this configuration, the first tube 32 can be softer than the second tube 33, enabling higher elasticity.

As the material forming the first resin layer 331, relatively soft known resin can be used, and for example, a urethane, polyurethane, silicon, or vinyl chloride material having a low hardness can be used. As the material forming the second resin layer 332, for example, a urethane, polyurethane, silicon, vinyl chloride material having high hardness can be used.

When urethane or polyurethane is used, the surface may be provided with hydrophilic coating. With this configuration, the catheter tube 31 can have high surface lubricity and can be easily inserted into the living body, whereby operability is improved, and the blood vessel wall can be prevented from being damaged. Furthermore, attachment of blood and proteins is less likely to occur, and thus formation of a blood clot can also be expected to be prevented.

The distal end tip 41 is fixed to the distal end of the first tube 32. As illustrated in FIG. 6, the distal end tip 41 has a tapered shape with the diameter gradually decreasing toward the distal end side. As illustrated in FIG. 6, the distal end tip 41 includes a base portion 49 inserted into the distal end of the first tube 32, the plurality of through holes 46 provided on the side surface, and the through hole 47 provided at the distal end of the distal end tip 41. The through holes 46 and 47 function as holes for blood removal. The through hole 47 of the distal end tip 41 is configured to communicate with the lumen 30A of the catheter 30. The distal end tip 41 can be formed of, for example, hard plastic or the like.

Further, with the hard distal end tip 41 fixed to the distal end portion of the first tube 32, the first tube 32 can be prevented from being crushed during blood removal.

The flat receiving surface 48 (corresponding to “contact portion”) that comes into contact with the flat surface 50a of the stylet 50 used before the insertion of the catheter 30 into the living body is formed on the inner side of the distal end tip 41, as illustrated in FIG. 5. As will be described later, with the distal end of the stylet 50 being in contact with the receiving surface 48, the catheter 30 expands in the longitudinal direction.

The clamp tube 34 is provided on the proximal end side of the second tube 33. A lumen into which the stylet 50 can be inserted can be provided on the inner side of the clamp tube 34. The clamp tube 34 can be formed using the same material as the catheter tube 31.

The catheter connector 35 connects the second tube 33 and the clamp tube 34 to each other. A lumen into which the stylet 50 can be inserted can be provided on the inner side of the catheter connector 35.

The straight connector 36 is provided on the proximal end side of the catheter 30. The straight connector 36 is sturdily affixed to the proximal end side of the clamp tube 34 (e.g., clamp tube 34 grips a distal end of straight connector 36 so that no sliding or rotation occurs between them). The straight connector 36 has a circular shape in a cross section intersecting the longitudinal direction in the proximal end portion. A lumen into which the stylet 50 can be inserted can be provided on the inner side of the straight connector 36. The straight connector 36 has an outer circumference provided with a male screw portion 36A (corresponding to “engagement portion”) that can engage with a female screw portion 53C of the stylet 50. The straight connector 36 is configured to be capable of being coupled to the stylet 50 by means of the male screw portion 36A.

(Stylet)

The stylet 50 includes a stylet tube 51 extending in the axial direction, and a stylet hub 52 (corresponding to a “hub”) to which the proximal end of the stylet tube 51 is fixed as illustrated in FIG. 2. The stylet 50 includes a coupling member 53 provided on the distal end side of the stylet hub 52 and coupling the stylet hub 52 and the straight connector 36 to each other.

The stylet tube 51 is a relatively rigid elongated body that extends in the axial direction. The stylet tube 51 is configured to be capable of being inserted into the lumen 30A of the catheter 30. The total length of the stylet tube 51 along the axial direction is configured to be longer than the total length (in its natural, relaxed state) of the catheter 30 along the axial direction. The stylet tube 51 includes a guide wire lumen 54 through which a guide wire (not illustrated) can be inserted (see FIG. 5). The stylet tube 51 is inserted into the living body together with the catheter 30 while being guided by the guide wire. The stylet tube 51 is removed from the catheter 30 by pulling out the stylet hub 52 toward the proximal end side, after the catheter 30 has been placed in the living body.

The distal end of the stylet tube 51 includes the flat surface 50a that comes into contact with the receiving surface 48 of the distal end tip 41 as illustrated in FIG. 5. The stylet tube 51 has a relatively high rigidity, and has a stiffness to enable pushing force toward the distal end side, applied by an operation with the hand, to be transmitted to the distal end tip 41. Thus, the stylet tube 51 has a function of expanding the catheter tube 31 in the longitudinal direction and dilating a narrow blood vessel, by bringing the flat surface 50a of the stylet tube 51 to contact with the receiving surface 48 of the distal end tip 41 and pushing the distal end tip 41 toward the distal end side.

The stylet hub 52 is arranged on the proximal end side of the catheter 30 when the catheter 30 is coupled to the stylet 50. As illustrated in FIG. 7, the stylet hub 52 includes an extending portion 52A extending along a first enclosing portion 53B of the coupling member 53 described later, on the outer side of the stylet hub 52 in the circumferential direction. The extending portion 52A is formed to have a hollow circular shape in a cross section intersecting the longitudinal direction which receives a proximal end of the straight connector 36.

As illustrated in FIG. 7, the coupling member 53 includes the first enclosing portion 53B that extends in the longitudinal direction so as to enclose a section between the attachment portion 53A attached to the stylet hub 52 and the male screw portion 36A (engagement portion). The extending portion 52A extends along the first enclosing portion 53B and comes into contact with the male screw portion 36A (engagement portion). When the male screw portion 36A and the extending portion 52A come into surface contact with each other, a sealing portion 55 sealing the proximal end side of the catheter 30 is formed between the catheter 30 and the stylet 50.

The coupling member 53 includes the female screw portion 53C that is provided on the distal end side of the first enclosing portion 53B and is screwed with the male screw portion 36A. The female screw portion 53C is configured to be engageable with the male screw portion 36A by rotating the coupling member 53 relatively with respect to the straight connector 36.

<How Catheter is Used>

Next, how the catheter assembly 100 described above is used will be described. FIG. 2 illustrates a state before the stylet tube 51 of the stylet 50 is inserted into the lumen 30A of the catheter 30, and FIG. 4 illustrates a state after the stylet tube 51 has been inserted into the lumen 30A of the catheter 30.

An operator such as a physician first inserts the stylet tube 51 of the stylet 50 into the lumen 30A of the catheter 30 as illustrated in FIG. 4. As a result, the stylet tube 51 passes through the inside of the straight connector 36, the clamp tube 34, the catheter connector 35, the second tube 33, and the first tube 32 in this order. Then, the flat surface 50a of the stylet tube 51 comes into contact with the receiving surface 48 of the distal end tip 41 (see FIG. 5).

The total length of the stylet tube 51 along the axial direction is configured to be longer than the total length of the catheter 30 along the axial direction as illustrated in FIG. 2. Thus, the distal end tip 41 is pressed toward the distal end side, with the flat surface 50a of the stylet tube 51 being in contact with the receiving surface 48 of the distal end tip 41. As a result, the distal end of the first tube 32 fixed to the distal end tip 41 is pulled toward the distal end side.

Thus, the catheter 30 receives force in the expanding direction, and the first tube 32, which is a relatively elastic portion of the catheter 30, expands in the axial direction. When the first tube 32 expands in the axial direction, the outer diameter of the first tube 32 decrease to be substantially the same as the outer diameter of the second tube 33. With the first tube 32 expanding in the axial direction, the proximal end of the catheter 30 and the stylet hub 52 are fixed to each other. The proximal end of the catheter 30 and the stylet hub 52 are fixed to each other, when the female screw portion 53C and the male screw portion 36A engage with each other due to a manual rotation of the coupling member 53.

Next, the operator inserts the catheter 30 through which the stylet tube 51 is inserted, along a guide wire (not illustrated) that has been inserted into the target portion in the living body in advance. In this processing, by inserting the stylet 50 into the catheter 30, the outer diameter of the first tube 32 becomes substantially the same as the outer diameter of the second tube 33. Therefore, the catheter 30 can be inserted into the living body in a minimally invasive manner. The operator inserts the catheter 30 into the living body until the through holes 46 and 47 of the distal end tip 41 are placed in the right atrium and the side hole 63 is placed in the inferior vena cava, and holds the catheter 30 in this state. In a state where the through holes 46 and 47 and the side hole 63 are arranged in the blood removal targets, the first tube 32 is arranged in the inferior vena cava which is a relative thick blood vessel, and the second tube 33 is arranged in the femoral vein which is a relatively thin blood vessel.

The operator then removes the stylet tube 51 and the guide wire from the catheter 30 by unscrewing coupling member 53 from straight connector 36. In this process, the stylet tube 51 and the guide wire are first pulled out to a position of the straight connector 36 of the catheter 30, temporarily clamped by forceps (not illustrated), and then is completely removed from the catheter 30. When the stylet tube 51 is removed from the lumen 30A of the catheter 30, the catheter 30 is released from force in a direction for causing it to expand in the axial direction, received from the stylet 50. Therefore, the first tube 32 contracts in the axial direction, and the outer diameter and inner diameter of the first tube 32 increase. Thus, the pressure loss in the first tube 32 can be reduced.

Next, the operator connects the straight connector 36 of the catheter 30 to the blood removal tube 11 of the extracorporeal circulatory device 1 illustrated in FIG. 1. Once completion of the connection of the catheter on the blood transmission is confirmed, the forceps of the clamp tube 34 are released and extracorporeal circulation starts.

When the extracorporeal circulation ends, the operator removes the catheter 30 from the blood vessel and performs hemostatic repair on the insertion site through a surgical procedure if necessary.

As described above, the catheter assembly 100 according to the present embodiment includes the catheter 30 and the stylet 50, and the sealing portion 55 sealing the proximal end side of the catheter 30 is provided between them. The catheter 30 includes the catheter tube 31, the receiving surface 48, and the straight connector 36. The catheter tube 31 is formed to have an elongated shape, is provided with the lumen 30A in which the blood can flow, and is configured to be expandable and contractible. The receiving surface 48 is provided in the distal end portion of the catheter tube 31, and enables the catheter tube 31 to expand in the longitudinal direction by the contacting. The straight connector 36 is provided on the proximal end side of the catheter 30 and is configured to be capable of being coupled with the stylet 50. The straight connector 36 includes the male screw portion 36A that can be engaged with the stylet 50. The stylet 50 is configured to be capable of being inserted into the lumen 30A, allows the catheter tube 31 to expand in the longitudinal direction by coming into contact with the receiving surface 48, and includes the stylet hub 52 and the coupling member 53. The stylet hub 52 is arranged on the proximal end side of the catheter 30. The coupling member 53 couples the stylet hub 52 and the straight connector 36 to each other, and is configured to be engageable with the male screw portion 36A.

The sealing portion 55 sealing the proximal end side of the catheter 30 is provided between the catheter 30 and the stylet 50, whereby sealing between the catheter 30 and the stylet 50 is reliably obtained.

The coupling member 53 includes the first enclosing portion 53B that extends in the longitudinal direction so as to enclose a section between the attachment portion 53A attached to the stylet hub 52 and the male screw portion 36A of the straight connector 36. The stylet hub 52 includes the extending portion 52A formed in a circular shape in a cross section intersecting the longitudinal direction and extending along the first enclosing portion 53B on the outer side of the stylet hub 52 in the circumferential direction. The sealing portion 55 can be formed by surface contact between the male screw portion 36A and the extending portion 52A.

### First Modification

Next, a catheter assembly 100A according to a first modification will be described with reference to FIG. 8. FIGS. 8 to 12 are diagrams used for a description on a main part of the catheter assembly 100A according to the first modification.

As illustrated in FIG. 8, the catheter assembly 100A according to the first modification is different from the first embodiment in that it includes a straight connector 36B forming a catheter 30B and a stylet hub 520 forming a stylet 50A. The other configurations are substantially the same as those in the first embodiment, and the description to be redundant herein is basically omitted.

The straight connector 36B has a proximal end portion 36C that is formed more linearly than the proximal end portion of the straight connector 36. The straight connector 36B has a circular shape in a cross section intersecting the longitudinal direction in the proximal end portion 36C, as in the case of the straight connector 36 of the first embodiment.

The stylet hub 520 includes the second enclosing portion 520A capable of surrounding the proximal end portion 36C of the straight connector 36, on the outer circumference of the straight connector 36. The second enclosing portion 520A is configured to be capable of coming into contact with the outer side surface of the proximal end portion 36C of the straight connector 36B.

The second enclosing portion 520A and the outer side surface of the proximal end portion 36C of the straight connector 36B may be in surface contact with each other while being inclined in substantially the same manner with respect to the longitudinal direction as illustrated in FIG. 9 and FIG. 10, and thus a sealing portion 550 sealing the proximal end side of the catheter 30 may be formed.

This configuration should not be construed in a limiting sense. For example, a second enclosing portion 520C of the stylet hub 520B and the outer side surface of the proximal end portion 36C of the straight connector 36B may be different from each other in the inclination with respect to the longitudinal direction as illustrated in FIG. 11 and FIG. 12. In this case, at least one of the two is deformed at a portion with different inclinations so that they are in circumferential surface contact with each other around the longitudinal direction. Thus, a sealing portion 550A is formed at the portion of the surface contact.

As described above, the straight connector 36 has a circular shape in a cross section intersecting the longitudinal direction in the proximal end portion in the first modification. The stylet hub 52 includes the second enclosing portion 520A capable of surrounding the proximal end portion of the straight connector 36, on the outer circumference of the straight connector 36, and capable of coming into contact with the outer side surface of the proximal end portion of the straight connector 36. The sealing portion 550 can also be formed by surface contact between the second enclosing portion 520A and the outer side surface of the straight connector 36.

When the sealing portion 550 is formed with the second enclosing portion 520A being in contact with the outer side surface of the proximal end portion 36C of the straight connector 36B as in the present first modification, no contact occurs on the inner circumference surface of the straight connector 36B. Thus, when surface treatment such as polymer coating is provided on the inner circumference surface, such coating or the like can be prevented from peeling. The same applies to the sealing portion 55 in the first embodiment.

### Second Modification

Next, a catheter assembly 100B according to a second modification will be described with reference to FIG. 13. FIG. 13 is a diagram used for a description on a main part of the catheter assembly 100B according to the second modification.

The catheter assembly 100B according to the second modification is, like the first modification, different from the first embodiment in that it includes a straight connector 36D forming a catheter 30C and a stylet hub 521 forming a stylet 50B. Further, the straight connector 36D of the second modification has a proximal end portion provided with a tapered surface 36E formed in a tapered shape. The other configurations are substantially the same as in the first embodiment.

The stylet hub 521 includes a contact surface 521A capable of coming into contact with the tapered surface 36E, and the sealing portion 551 sealing the proximal end side of the catheter 30 is configured by surface contact between the tapered surface 36E and the contact portion 521A.

As described above, the straight connector 36D in the second modification has the proximal end portion provided with the tapered surface 36E formed in a tapered shape. The stylet hub 521 has the contact surface 521A capable of coming into contact with the tapered surface 36E of the straight connector 36D. The sealing portion 551 can reliably obtain the sealing property as in the first embodiment or the like due to the surface contact between the tapered surface 36E and the contact surface 521A.

### Fourth Embodiment

Next, the catheter assembly 200 according to a fourth embodiment of the present invention will be described with reference to FIGS. 14 to 16. FIGS. 14 to 16 are diagrams used for a description on the configuration of the catheter assembly 200 according to the fourth embodiment. The catheter assembly 200 according to the present embodiment has the percutaneous catheter (hereinafter referred to as “catheter”) 60 different from that in the third embodiment.

This catheter 60 is what is known as a double lumen catheter configured to simultaneously enable both transmission and removal of blood. Thus, in the present embodiment, the catheter 60 is in charge of functions of two catheters, that is, the venous catheter (blood removal catheter) 5 and the arterial catheter (blood transmission catheter) 6 in the extracorporeal circulatory device 1 in FIG. 1.

The catheter 60 according to the present embodiment is different from the catheter 30 according to the first embodiment in that a double tube structure is provided as illustrated in FIGS. 15 and 16. In such a structure, a third tube 161 having a first lumen 61 in communication with a blood transmission side hole 163 is provided inside a lumen of the second tube 33.

With the catheter 60, the blood is removed from a vein (vena cava) of the patient with a pump of the extracorporeal circulatory device 1 activated. Thereafter, gas exchange inside the blood is performed with the artificial lung 2 to oxygenate the blood, and then the resultant blood is returned to the vein (vena cava) of the patient again. In this manner, the Veno-Venous (VV) procedure can be performed.

Hereinafter, each configuration of the catheter 60 will be described. The parts common to the first embodiment will be omitted, and characteristic parts of the present embodiment will be described. The parts having the same functions as those in the first embodiment are designated by the same reference numerals, and the description thereof will be omitted.

As illustrated in FIG. 15, the catheter 60 includes the first tube 32, the second tube 33, the distal end tip 41 that is provided at the distal end of the first tube 32 and includes the through holes 46 and 47, and the third tube 161 provided inside the second tube 33.

As illustrated in FIG. 15, the catheter 60 includes the first lumen 61 that functions as the blood transmission channel and a second lumen 62 that functions as the blood removal channel.

The first lumen 61 is formed inside the third tube 161. The second lumen 62 is formed through the first tube 32 and the second tube 33, from the distal end to the proximal end.

The second tube 33 includes the blood transmission side hole 163 in communication with the first lumen 61 that is the blood transmission channel and a blood removal side hole 164 in communication with the second lumen 62 that is the blood removal channel. The blood transmission side hole 163 and the blood removal side hole 164 are formed to have an elliptical shape, but are not limited this shape.

The third tube 161 is configured to be inserted into the second lumen 62 from the proximal end side of the second tube 33 and to be in communication with the blood transmission side hole 163.

The blood transmission side hole 163 is arranged in the blood transmission target in the living body. The blood oxygenated by the artificial lung 2 is transmitted into the living body through the blood transmission side hole 163.

The through holes 46 and 47 of the distal end tip 41 and blood removal side hole 164 are arranged in different blood removal targets in the living body so that blood can be efficiently removed. When the through hole 46, 47 or the blood removal side hole 164 is closed as a result of being adsorbed on a blood vessel wall, blood can be removed through unclosed one of the holes, whereby extracorporeal circulation can be stably performed.

In the present embodiment, the catheter 60 is inserted from the internal jugular vein of the neck, passes through the superior vena cava and the right atrium, to have the distal end placed in the inferior vena cava. The blood transmission target is the right atrium, and the blood removal target includes two portions that are the superior vena cava and the inferior vena cava.

As illustrated in FIGS. 14 and 15, the catheter 60 is inserted, in a state where the stylet 50 is inserted, into the living body and is held once the through holes 46 and 47 of the distal end tip 41 are placed in the inferior vena cava and the blood removal side hole 164 is placed in the internal jugular vein.

As in the first embodiment, the first tube 32 is configured to have an inner diameter that is larger than that of the second tube 33. In a state where the through holes 46 and 47 and the side hole 63 are arranged in the blood removal targets, the first tube 32 is placed in the inferior vena cava which is a relatively thick blood vessel, and the second tube 33 is placed in the femoral vein which is a relatively thin blood vessel.

As illustrated in FIG. 15, a straight connector 136 includes a first straight connector 137 and a second straight connector 138. The first straight connector 137 is configured to communicate with the first lumen 61, and the second straight connector 138 is configured to communicate with the second lumen 62. The straight connector 136 is configured as a Y-shaped Y connector formed with the first straight connector 137 branched from the second straight connector 138. For the sake of illustration, in FIG. 15, the first straight connector 137 and the second straight connector 138 are close to each other, but the first straight connector 137 and the second straight connector 138 are actually arranged to be more separated from each other by an amount larger than that illustrated in FIG. 15.

The first straight connector 137 is coupled to the proximal end portion of the third tube 161. The second straight connector 138 is coaxially coupled to the proximal end portion of the second tube 33. A blood transmission tube (blood transmission line) is connected to the first straight connector 137, and a blood removal tube (blood removal line) is connected to the second straight connector 138. The first straight connector 137 is provided with a male screw portion 137A, and the second straight connector 138 is provided with a male screw portion 138A.

The first tube 32 is configured to function in the same manner as in the first embodiment. When the stylet 50 is inserted into the catheter 60, as illustrated in FIG. 16, the first tube 32 expands to have the outer diameter and the inner diameter reduced. As a result, the catheter 60 can be inserted into the living body in a minimally invasive manner. When the stylet 50 is removed from the catheter 60, the first tube 32 is contracted in the axial direction to have the inner diameter increased as illustrated in FIG. 14. Thus, the pressure loss inside the first tube 32 can be reduced.

As described above, the catheter assembly 200 of the present embodiment has the catheter 60. Thus, both blood removal and blood transmission functions can be provided with a single catheter.

The present invention is not limited to the above-described embodiments, and various modifications can be made within the scope of the claims. The embodiments and the like in which the catheter 30 includes the straight connector 36 have been described above. However, the type of the connector is not limited to the straight connector as long as a sealing portion can be formed and the connector can be connected to a mating component.

What is claimed is:

1. A catheter assembly comprising:
a catheter including a tube that is formed to have an elongated shape with a lumen in which blood is flowable, wherein at least a portion of the tube is expandable and contractible, wherein the catheter has a first contact portion provided at a distal end portion of the catheter which enables the tube to expand in a longitudinal direction;
a stylet that is configured to be inserted in the lumen with a stylet tube that comes into contact with the first contact portion to expand the tube in the longitudinal direction; and
a connector affixed to a proximal end side of the tube, wherein the connector includes a first engagement portion;
wherein the stylet includes a hub disposed on the proximal end side of the stylet tube and a coupling member that couples the hub and the connector to each other, wherein the coupling member is engageable with the first engagement portion by rotation, and wherein the coupling member rotates independently of the hub.

2. The catheter assembly according to claim 1, wherein the coupling member includes an attachment portion that is rotatably attached to the hub, and wherein the attachment portion is arranged to be separated from an outer surface of the hub in a radial direction.

3. The catheter assembly according to claim 2, wherein:
the coupling member includes a second engagement portion that is engageable with the first engagement portion through screwing; and
the attachment portion applies force to fix the hub to the connector in the longitudinal direction, through the screwing.

4. The catheter assembly according to claim 2, wherein:
the coupling member further includes a second engagement portion that is engageable with the first engagement portion, and a first enclosing portion that extends in the longitudinal direction so as to enclose a section between the attachment portion and the second engagement portion;
the hub includes a second contact portion that comes into contact with the connector on a proximal end side; and
the second contact portion is arranged to be separated from the first enclosing portion in the radial direction.

5. The catheter assembly according to claim 1, wherein:
the connector has a proximal end portion provided with a tapered surface formed in a tapered shape;
the hub includes a contact surface configured to come into contact with the tapered surface; and
contact between the tapered surface and the contact surface forms a sealing portion which seals the proximal end side of the catheter.

6. A catheter assembly for extracorporeal blood flow, comprising:
a catheter tube section extending from a proximal end to a distal end and having an internal lumen, wherein the catheter tube section has an elastic portion with an expanded diameter when in a relaxed state;
a distal tip affixed to the distal end of the catheter tube section, wherein the distal tip includes a contact portion accessible via the lumen;
a straight connector affixed to the proximal end of the catheter tube section, wherein the straight connector includes a first engagement portion; and
a stylet including a stylet tube configured to be inserted through the lumen to the contact portion in order to longitudinally expand the elastic portion, wherein the stylet further includes a hub at a proximal end of the stylet tube, and wherein the stylet further includes a coupling member mounted for rotation on the hub and having a second engagement portion configured to rotationally couple to the first engagement portion at a position where the stylet tube longitudinally expands the elastic portion, wherein the coupling member is configured to rotate onto the straight connector without transferring rotation to the hub.

7. The catheter assembly of claim 6, wherein the hub includes a notched surface rotationally receiving an attachment portion of the coupling member with a gap between the notched surface and the attachment portion.

8. The catheter assembly of claim 7, wherein the second engagement portion screws onto the first engagement portion to cause the attachment portion to press against a longitudinal side of the notched surface, and wherein the stylet tube transfers a pressing force to the contact portion of the distal tip.

9. The catheter assembly of claim 8, wherein the straight connector includes a first sealing surface portion, wherein the hub includes a second sealing surface portion, and wherein when the second engagement portion screws onto the first engagement portion then the first and second sealing surface portions come into sealing contact to seal the catheter assembly.
